(19) United States
(12) Patent Application Publication
(10) Pub. No.: US 2024/0212834 A1
(43) Pub. Date: Jun. 27, 2024

(54) Method And Apparatus for Evaluating Proficiency in Time Marking Section

(71) Applicants: Kyu Eun Lee, Seoul (KR); Hyoun-Joong Kong, Seoul (KR); Su-jin Kim, Seoul (KR); Hoorang Shin, Jeju-si (KR)

(72) Inventors: Kyu Eun Lee, Seoul (KR); Hyoun-Joong Kong, Seoul (KR); Su-jin Kim, Seoul (KR); Hoorang Shin, Jeju-si (KR)

(21) Appl. No.: 18/103,296

(22) Filed: Jan. 30, 2023

(30) Foreign Application Priority Data
Dec. 23, 2022 (KR) 10-2022-0183265

(51) Int. Cl. G16H 40/20; G16H 40/67

(52) U.S. Cl. CPC G16H 40/20; G16H 40/67

(57) ABSTRACT

Disclosed are a method and an apparatus for evaluating proficiency according to a length of a timemarking section. The present embodiment provides a method and apparatus for evaluating proficiency according to a length of a timemarking section to evaluate proficiency of a medical person who performs a medical procedure from a medical video based on a result of the medical video analyzed based on a proficiency evaluation index predefined in the medical video.

### BACKGROUND OF THE INVENTION

### 1. Field of the Invention

One embodiment of the present invention relates to a method and an apparatus for evaluating proficiency according to a length of a timemarking section.

### 2. Description of the Related Art

The statements in this section merely provide background information related to the present embodiment and do not necessarily constitute related art.

Surgeons generally undertake extensive study before performing a surgical procedure. Typically, surgeons study using generic anatomical models, such as photographs or drawings. More recently, various pre-operative diagnostic procedures (e.g., x-ray, CT, MRI, etc.) have been performed using patient-specific anatomical information.

It is preferable to make additional anatomic and surgical procedure information available to surgeons. A surgeon planning surgery on a particular patient is provided with a surgical site video recording of an earlier surgical procedure performed on the particular patient. A surgeon is provided with one or more surgical video recordings of surgical procedures on other patients that are similar to the surgical procedure planned for a particular patient. Such information is provided to a surgeon prior to the surgeon undertaking a particular surgical procedure. The above information is intraoperatively provided to a surgeon.

A video database is configured to include intraoperative surgical site video recordings of various procedures undergone by various patients. A medical device capable of recording a video is configured to further include an input that enables a surgeon using the medical device to highlight and annotate the video recording in real-time as it is being recorded. It is preferable to provide a computer-based pattern matching algorithm to search through the individual records of the video database, identify relevant video records, and provide a surgeon with this relevant information for a particular surgical procedure.

### SUMMARY OF THE INVENTION

### Technical Problem

The present embodiment is to provide a method and apparatus for evaluating proficiency according to a length of a timemarking section to evaluate proficiency of a medical person who performs a medical procedure from a medical video based on a result of the medical video analyzed in accordance with on a proficiency evaluation index predefined in the medical video.

### Technical Solution

According to one aspect of the present embodiment, there is provided an apparatus for evaluating proficiency including: a keyword table that stores proficiency evaluation indices preset for each type of surgery; an image stream unit that receives a stream image for a specific surgery; a stream image section division unit that divides the stream image into a plurality sections; and a proficiency evaluation unit that extracts the proficiency evaluation indices corresponding to each of the plurality of sections for the stream image divided into the plurality of sections, and generates proficiency evaluation data according to satisfaction for the proficiency evaluation indices for each of the plurality of sections.

### Advantageous Effects

According to the present embodiment as described above, it is possible to evaluate proficiency of the medical person who performs a medical procedure from the medical video based on a result of the medical video analyzed in accordance with the proficiency evaluation index predefined in the medical video.

### BRIEF DESCRIPTION OF THE DRAWINGS

FIG. 1 is a view schematically showing a proficiency evaluation system according to the present embodiment.

FIG. 2a is a view showing a proficiency evaluation apparatus according to the present embodiment.

FIG. 2b is a view schematically showing an operation process of a proficiency evaluation unit according to the present embodiment.

FIG. 3a is a view for describing a method for providing timemarking based on speech recognition and a tag according to the present embodiment.

FIG. 3b is a view showing a method for analyzing an influence and evaluating proficiency according to a length of a timemarking section according to the present embodiment.

FIG. 4 is a view showing a keyword table according to the present embodiment.

FIG. 5 is a view showing selection of a text corresponding to an audio and an audio-based keyword coincident with keywords stored in the keyword table according to the present embodiment.

FIG. 6 is a view showing control of each section of a stream image according to the present embodiment.

FIGS. 7a to 7d are views showing tags corresponding to scenes of a reference image according to the present embodiment.

FIG. 8 is a view showing an influence table (proficiency evaluation table) according to the present embodiment.

### DETAILED DESCRIPTION OF THE INVENTION

Hereinafter, the present embodiment will be described with reference to accompanying drawings.

FIG. 1 is a view schematically showing a timemarking system according to the present embodiment.

The timemarking system according to the present embodiment includes an image transmission apparatus 110 and a proficiency evaluation apparatus 120. Components of the timemarking system are not necessarily limited thereto.

The image transmission apparatus 110 is preferably installed in an operating room, but is not necessarily limited thereto. The image transmission apparatus 110 captures a surgery image in the operating room in real-time, and transmits the surgery image to an image stream unit 220, and at the same time, stores the surgery image in a surgery image DB 210. The image transmission apparatus 110 transmits the surgery image obtained by capturing a surgical procedure to the proficiency evaluation apparatus 120. Proficiency may be evaluated in real-time through a video transmitted by the proficiency evaluation apparatus 120.

The proficiency evaluation apparatus 120 selects a reference image that satisfies a preset condition in a category which is the same as that of the stream image. The proficiency evaluation apparatus 120 selects an audio-based keyword that matches a text corresponding to an audio of the steam image and keywords stored in a keyword table 244. The proficiency evaluation apparatus 120 selects, as scene-based keywords, a tag corresponding to a scene of the stream image and a tag that matches a tag pre-stored in the reference image. The proficiency evaluation apparatus 120 determines a section keyword representing the section for each section by applying a preset weight after mapping the audio-based keyword and scene-based keyword of the stream image for each section.

The proficiency evaluation apparatus 120 time-marks the section keyword for each section. When the section keyword is selected, the proficiency evaluation apparatus 120 allows the image to move the corresponding section and plays the image. For example, the proficiency evaluation apparatus 120 time-marks a surgery image with longer than 10 hours by determining a section keyword for each section with respect to the surgery image.

The proficiency evaluation apparatus 120 generates proficiency evaluation data for a medical person who performs a medical operation in a medical video by analyzing the medical video based on predefined proficiency evaluation indices for the medical video.

The proficiency evaluation apparatus 120 stores proficiency evaluation indices including evaluation items and evaluation criteria for evaluating the proficiency of a medical person when the proficiency for surgery of the medical person is evaluated by analyzing the medical video (in particular, the surgery video). The proficiency evaluation apparatus 120 pre-stores an influence table. The influence table stores the proficiency evaluation indices for evaluating the proficiency.

The proficiency evaluation apparatus 120 time-marks a section corresponding to the preset keyword in the surgery video. The proficiency evaluation apparatus 120 determines a section corresponding to a list or combination of consecutive keywords related to surgery in the surgery video. The proficiency evaluation apparatus 120 performs timemarking on the section corresponding to the keyword. The proficiency evaluation apparatus 120 evaluates the proficiency of the medical person who performs surgery by analyzing a timemarking section.

In other words, the proficiency evaluation apparatus 120 determines a section corresponding to the keyword among a ‘first section’, a ‘second section’, and a ‘third section’ in surgery video ‘A’. The proficiency evaluation apparatus 120 gives a score obtained by evaluating proficiency for each of the ‘first section’, the ‘second section’, and the ‘third section’. In this case, a proficiency qualitative evaluation score may be received. The proficiency evaluation apparatus 120 calculates a final evaluation result by finally summing up scores for each section or summing up the score for each section and the received qualitative evaluation score.

The proficiency evaluation apparatus 120 pre-defines evaluation items and evaluation criteria in the influence table in order to evaluate proficiency for each section according to each keyword. The proficiency evaluation apparatus 120 evaluates the section for each keyword with reference the influence table. For example, the proficiency evaluation apparatus 120 confirms an average surgery time (e.g.: 10 minutes) from among the evaluation items when proficiency for a section corresponding to keyword ‘A’ is evaluated. The proficiency evaluation apparatus 120 extracts the average surgery time from among the preset evaluation items in the influence table.

The proficiency evaluation apparatus 120 determines the section corresponding to keyword ‘A’ in the medical video. The proficiency evaluation apparatus 120 measures a surgery duration of time of the section corresponding to keyword ‘A’ in the medical video. The proficiency evaluation apparatus 120 compares the average surgery time among the evaluation items with the surgery duration of time of the section corresponding to keyword ‘A’ in the medical video. The proficiency evaluation apparatus 120 evaluates the proficiency for the medical person according to whether the surgery duration of time of the section corresponding to keyword ‘A’ in the medical video is within the average surgery time.

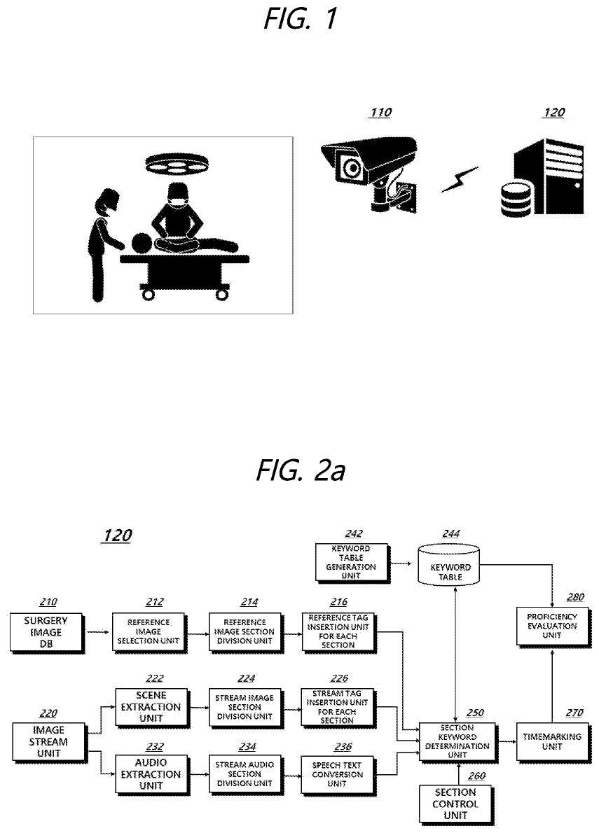

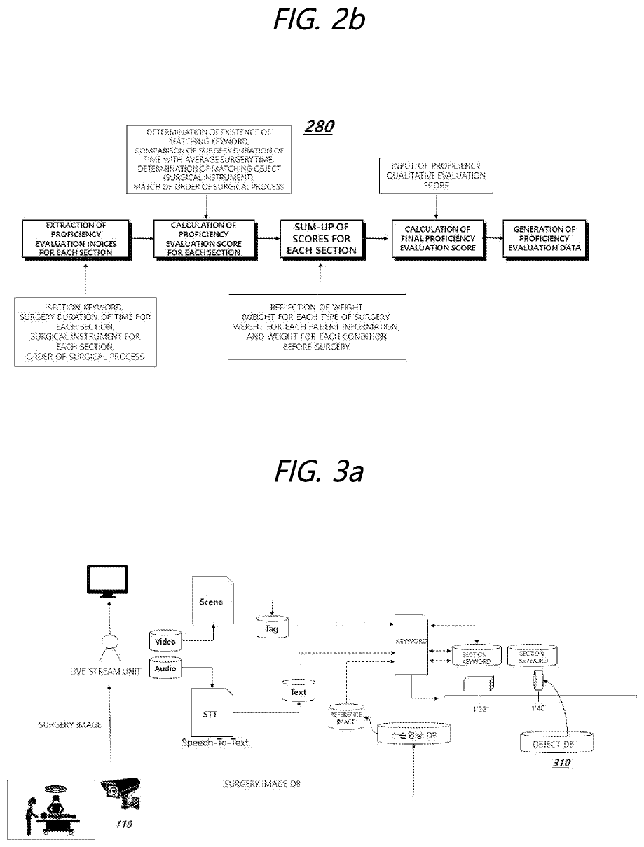

FIG. 2a is a view showing a proficiency evaluation apparatus according to the present embodiment.

The proficiency evaluation apparatus 120 according to the present embodiment includes a keyword table generation unit 242, the keyword table 244, the surgery image DB 210, a reference image selection unit 212, a reference image section division unit 214, a reference tag insertion unit 216 for each section, the image stream unit 220, a scene extraction unit 222, a stream image section division unit 224, a stream tag insertion unit 226 for each section, an audio extraction unit 232, a stream audio section division unit 234, a speech text conversion unit 236, a section keyword determination unit 250, a section control unit 260, and a timemarking unit 270. Components included in the proficiency evaluation apparatus 120 are not necessarily limited thereto.

Each component included in the proficiency evaluation apparatus 120 may be connected to a communication path for connecting software modules or hardware modules in the apparatus and may organically operate with each other. These components communicate with each other using one or more communication buses or signal lines.

Each component of the proficiency evaluation apparatus 120 shown in FIG. 2a means a unit for processing at least one function or operation, and may be implemented as a software module, a hardware module, or a combination of software and hardware.

The keyword table generation unit 242 generates a predefined table and stores keywords for each type of surgery in the keyword table. The keyword table generation unit 242 matches a plurality of image objects for each keyword and stores the plurality of image objects in the object DB 310.

The keyword table 244 stores a preset keyword for each type of surgery. The keyword table 244 stores a table in which a plurality of keywords are predefined for each type of surgery, and stores the plurality of image objects by being matched for each keyword.

The keyword table 244 stores the preset proficiency evaluation indices (evaluation items and evaluation criteria) for each type of surgery. The keyword table 244 may extract proficiency evaluation indices for each section so that the proficiency for each section may be evaluated in each surgery. The proficiency evaluation index for each section may include at least one information about keywords of the corresponding surgery section, surgery duration of time of the corresponding surgery section, surgical instrument used in the corresponding surgery section, and order of a surgical process of the corresponding surgery section. However, the evaluation indices for evaluating the proficiency are not limited thereto.

The surgery image DB 210 records and stores the entire surgery image recorded by the image transmission apparatus 110. The surgery image DB 210 matches and stores surgery information, surgery type, surgery name, surgeon, surgery method, and patient information for each surgery image. The surgery image DB 210 stores surgery images by being classified into brain surgery, cancer surgery, cancer surgery, surgical surgery, robotic surgery, etc. according to the surgery type. The surgery image DB 210 stores a plurality of surgery images.

The reference image selection unit 212 confirms surgical conditions (surgery name, surgeon, surgery method, and patient information) of the stream image, and selects a reference image from among the plurality of reference images when the reference image has a preset number or more of surgical conditions being matched.

The reference image selection unit 212 selects any one image corresponding to a user command (surgery type, surgery name, surgeon, method, and patient surgery information) from among the plurality of surgery images as a reference image. The reference image selection unit 212 selects, according to the user command, an image that may be referred to by other people from among the plurality of surgery images stored in the surgery image DB 210. The reference image selection unit 212 selects a reference image corresponding to the input user command (patient age, patient gender, surgery method, surgery surgeon, and size of tumor in case of cancer surgery).

The reference image selection unit 212 provides a reference image for each type of surgery. The reference image selection unit 212 selects an image as a reference image when the image has a preset number or more of surgical conditions (surgery name, surgeon, surgery method, and patient information) of the plurality of surgeries by comparing the surgical conditions of the plurality of surgeries with surgery conditions of the stream image.

The reference image section division unit 214 divides the reference image into a plurality of sections. The reference image section division unit 214 divides a section of the reference image based on a preset unit time and a sequence number of each frame, or divides a section of the reference image by recognizing each scene of the reference image and grouping similar scenes into each section using an artificial intelligence.

The reference tag insertion unit 216 for each section inserts a tag corresponding to the reference image for each section of the reference image to generate a reference tag for each section.

The image stream unit 220 receives a stream image for a specific surgery. The image stream unit 220 outputs the surgery image received from the image transmission apparatus 110 in real-time using a display.

The scene extraction unit 222 separates only video data from the surgery image received from the image stream unit 220. The scene extraction unit 222 extracts a video from the stream image.

The stream image section division unit 224 divides the stream image into a plurality of sections. The stream image section division unit 224 divides a section of the stream image based on a preset unit time and a sequence number of each frame, or divides a section of the stream image by recognizing each scene of the stream image and grouping similar scenes into each section using an artificial intelligence.

The stream tag insertion unit 226 for each section inserts a tag corresponding to the scene of the video for each section to generate a stream tag for each section.

The audio extraction unit 232 extracts an audio from the stream image.

The stream audio section division unit 234 divides the audio of the stream image into a plurality of sections. The stream audio section division unit does not necessarily operate, but may selectively operate. The stream audio section division unit 234 divides a section of the stream image based on a preset unit time, or divides a section with similar contents by recognizing an audio-based text using an artificial intelligence.

The speech text conversion unit 236 separates only audio data from the surgery image received from the image stream unit 220. The speech text conversion unit 236 converts an audio into a text to generate an audio-based text.

The section keyword determination unit 250 determines an audio-based keyword based on the keywords stored in the keyword table 244 and an audio of the stream image. The section keyword determination unit 250 determines a scene-based keyword based on the reference image and a video of the stream image. The section keyword determination unit 250 determines a scene-based keyword based on the reference image and a stream tag for each section. The section keyword determination unit 250 determines a section keyword based on the audio-based keyword and scene-based keyword matched to a specific section.

The section keyword determination unit 250 confirms whether there is a keyword that matches the audio-based text among the keywords stored in the keyword table 244. When there are one or more keywords that match the audio-based text among the keywords stored in the keyword table 244, the section keyword determination unit 250 determines the keyword as an audio-based keyword.

The section keyword determination unit 250 compares the stream tag for each section with the reference tag for each section. The section keyword determination unit 250 determines, as a scene-based keyword, a tag that matches one of the stream tag for each section and the reference tag for each section.

The section keyword determination unit 250 matches the audio-based keyword and the scene-based keyword to each section. The section keyword determination unit 250 determines, as a section keyword, one of the audio-based keyword and the scene-based keyword by applying a weight to each of the audio-based keyword and the scene-based keyword matched to each section.

The section control unit 260 confirms a time when the audio-based keyword is selected from the stream image. The section control unit 260 resets, as a specific section, frames in which the image objects matched to the keywords stored in the keyword table exist from the time when the audio-based keyword is selected.

The timemarking unit 270 time-matches the section keyword to a specific section.

A proficiency evaluation unit 280 extracts proficiency evaluation indices corresponding to each of the plurality of sections for the stream image divided into a plurality of sections. The proficiency evaluation unit 280 generates proficiency evaluation data according to satisfaction of the proficiency evaluation indices for each of the plurality of sections.

The proficiency evaluation unit 280 gives a proficiency evaluation score obtained by evaluating the proficiency according to the satisfaction of the proficiency evaluation indices for each of the plurality of sections (e.g., first to twelfth sections). The proficiency evaluation unit 280 calculates a final proficiency evaluation score by finally summing up scores for each of the plurality of sections.

The proficiency evaluation unit 280 extracts one or more of the surgery duration of time, the surgical instrument, the section keyword, and occurrence of surgical complications when the proficiency is evaluated for each of the plurality of sections.

When there is a keyword coincident with the section keyword pre-stored in the keyword table 244 for each section among audio-based keywords or scene-based keywords extracted from the stream image for each of the plurality of sections, the proficiency evaluation unit 280 gives the medical person who performs surgery in the corresponding section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the stream image for each of the plurality of sections is within the average surgery time pre-stored in the keyword table 244 for each section.

The proficiency evaluation unit 280 gives the medical person who performs surgery in a specific section for a maximum proficiency evaluation score corresponding to the section keyword when the surgery duration of time is within the average surgery time. The proficiency evaluation unit 280 gives a proficiency evaluation score, which is obtained by subtracting a first score corresponding to the section keyword for the medical person who performs surgery in the specific section when the surgery duration of time exceeds the average surgery time by a first threshold (e.g.: 10 seconds) or less. The proficiency evaluation unit 280 gives a proficiency evaluation score, which is obtained by subtracting a second score corresponding to the section keyword for the medical person who performs surgery in the specific section when the surgery duration of time exceeds the average surgery time by a second threshold (e.g.: 20 seconds) or less.

The proficiency evaluation unit 280 selects a keyword coincident with the audio-based keyword or scene-based keyword extracted from the stream image for each of the plurality of sections among the section keywords pre-stored in the keyword table 244 for each section. The proficiency evaluation unit 280 extracts an image object matched to the keyword coincident with the audio-based keyword or the scene-based keyword. The proficiency evaluation unit 280 gives the medical person who performs surgery in the corresponding section for a score, which is equal to or higher than the preset threshold, as an evaluation score for the corresponding section when there is an object coincident with the surgical instrument pre-stored in the keyword table for each section among image objects.

When a plurality of indices are satisfied among the surgery duration of time, the surgical instrument, the section keyword, and the occurrence of surgical complications, the proficiency evaluation unit t 280 calculates a final proficiency evaluation score by summing up scores for each section after reflecting a weight to each satisfied index to provide an evaluation score for each section.

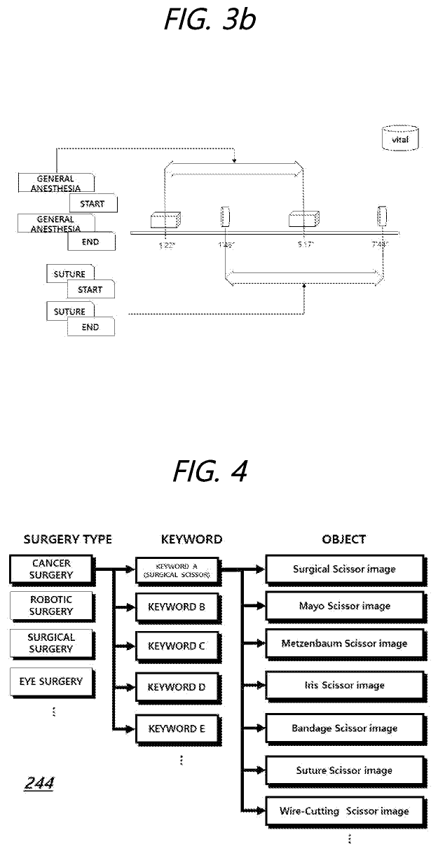

FIG. 3a is a view for describing a method for providing timemarking based on speech recognition and a tag according to the present embodiment.

The image transmission apparatus 110 streams a medical video including surgery name, surgeon, surgery method, and patient information.

The proficiency evaluation apparatus 120 extracts only a video from the medical video received from the image transmission apparatus 110 through streaming, and matches a tag for each section according to a specific scene in the image. The proficiency evaluation apparatus 120 extracts video data from the medical video. The proficiency evaluation apparatus 120 inserts a scene-based tag for the specific scene from the video data.

The proficiency evaluation apparatus 120 extracts only an audio from the medical video received from the image transmission apparatus 110 through streaming, and generates an audio-based text by converting an audio into a text. The proficiency evaluation apparatus 120 confirms whether there is a matching keyword by comparing a speech-based text with the predefined keyword table 244.

The proficiency evaluation apparatus 120 extracts a specific section of the speech-based text existing in the keyword table 244. The proficiency evaluation apparatus 120 time-marks the specific section. The proficiency evaluation apparatus 120 may use the keyword in combination with the text keyword and the visual object (e.g., surgical instrument) when the specific section of the speech-based text does not exist in the keyword table 244. In other words, after a surgical instrument, which may necessarily appear in an image when the text keyword appears, is stored in the form of an image, a section in which the surgical instrument is used may be specified through video analysis.

The proficiency evaluation apparatus 120 generates an audio-based keyword by comparing the pre-stored keyword table 244 with the audio-based text. The proficiency evaluation apparatus 120 generates a scene-based keyword by comparing a tag matched to each section according to the specific scene with a tag matched to each section according to a specific scene of the reference image. The proficiency evaluation apparatus 120 determine a section keyword representing the section by applying a preset weight to the audio-based keyword and the scene-based keyword matched to the specific section.

The proficiency evaluation apparatus 120 extracts a tag corresponding to a specific keyword, a text, and a section corresponding to the reference image. The proficiency evaluation apparatus 120 time-marks the section keyword for each section. The proficiency evaluation apparatus 120 outputs the section keyword together with the video in a time-marked section when the video is played. The proficiency evaluation apparatus 120 outputs the visual object (e.g., surgical instrument) matched to the keywords together with the video in the time-marked section when the video is played.

The visual object (e.g., surgical instrument) is pre-stored in the object DB 310 preset by a user. The proficiency evaluation apparatus 120 displays the input keyword together with a time mark on an upper side of a playback player only when the time mark matches the input keyword with a preset accuracy or higher.

The user may confirm a section keyword for each section using the visual object (e.g., surgical instrument). The user may grasp a progression state based on the visual object (e.g., surgical instrument). The user may receive an analysis service using the visual object (e.g., surgical instrument). When the user inputs (e.g., clicks) the visual object (e.g., surgical instrument), the proficiency evaluation apparatus 120 skips play of the corresponding section.

The proficiency evaluation apparatus 120 extracts a specific section corresponding to the preset keyword in the medical video (surgery video). The proficiency evaluation apparatus 120 performs timemarking on the specific section. The proficiency evaluation apparatus 120 plays the specific section corresponding to the keyword.

The proficiency evaluation apparatus 120 extracts a visual object (e.g.: surgical instrument) specified in the medical video. The proficiency evaluation apparatus 120 extracts a tag defining a specific scene in the video or the media stream. The proficiency evaluation apparatus 120 plays a keyword corresponding to the tag with timemarking data.

The proficiency evaluation apparatus 120 plays the keyword coincident with the tag with the timemarking data, so that each scene can be efficiently recognized. The proficiency evaluation apparatus 120 outputs the keywords that match the tags to each scene without a separate indexing process, so that an end user may quickly recognize and find a specific scene in an image that the user wants to find.

FIG. 3b is a view showing a method for analyzing an influence and evaluating proficiency according to a length of a timemarking section according to the present embodiment.

The proficiency evaluation apparatus 120 converts audio data input from the stream image into predefined metadata. The proficiency evaluation apparatus 120 generates an influence analysis result, which is obtained by analyzing an influence, by substituting the metadata, the timemarking data, and vital data in a preset influence table. The proficiency evaluation apparatus 120 compares reference data with user data to generate proficiency evaluation data obtained by evaluating proficiency of the user.

The proficiency evaluation apparatus 120 selects, as audio-based keywords, one or more keywords coincident with keywords in the keyword table 244 from ‘Now general anesthesia starts. Nurse, please connect a laryngeal tube to a patient . . . ’ which is said by the medical person who performs anesthesia in the corresponding section of the stream image when the general anesthesia is started.

The proficiency evaluation apparatus 120 selects, as audio-based keywords, one or more keywords coincident with keywords in the keyword table 244 from ‘Now, incision for *** starts . . . ’ which is said by the medical person who performs anesthesia in the corresponding section of the stream image when the general anesthesia ends.

The proficiency evaluation apparatus 120 selects, as audio-based keywords, one or more keywords coincident with keywords in the keyword table 244 from ‘Please finish suturing for *** in this way and treat . . . ’ which is said by the medical person who performs anesthesia in the corresponding section of the stream image when the general anesthesia ends.

The proficiency evaluation apparatus 120 selects, as audio-based keywords, one or more keywords coincident with keywords in the keyword table 244 from ‘The heat rate is returning to normal at *** . . . ’ which is said by the medical person who performs anesthesia in the corresponding section of the stream image when the general anesthesia ends.

FIG. 4 is a view showing a keyword table according to the present embodiment.

The keyword table generation unit 242 generates a predefined table and stores keywords for each type of surgery in the keyword table. For example, the keyword table generation unit 242 predefines keywords a, b, c, d, and e for cancer surgery among surgeries. The keyword table generation unit 242 matches a plurality of image objects for each keyword and stores the plurality of image objects in the object DB 310.

For example, assuming that ‘keyword a’ is ‘surgical scissor’, the keyword table generation unit 242 matches a surgical scissor image, a mayo scissor image, a metzenbaum scissor image, an iris scissor image, a bandage scissor image, a suture scissor image, and a wire-cutting scissor image to ‘surgical scissor’ (keyword a), and stores the matched result in the object DB 310.

The keyword table generation unit 242 selects a plurality of keywords for each type of surgery (cancer surgery, surgical surgery, eye surgery, or robotic surgery) in advance. The keyword table generation unit 242 maps an image object for each of the plurality of keywords and stores the image object in the object DB 310.

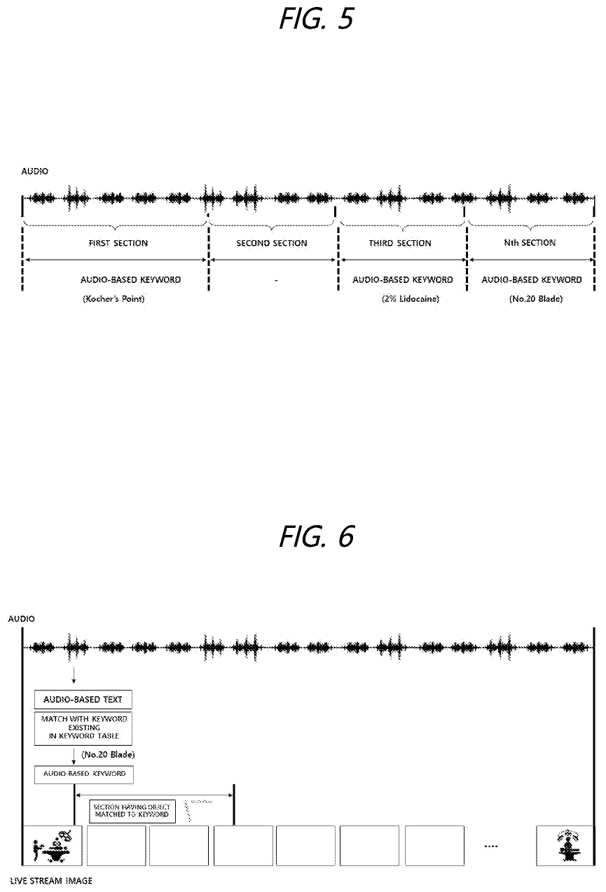

FIG. 5 is a view showing selection of a text corresponding to an audio and an audio-based keyword matching with keywords stored in the keyword table according to the present embodiment.

The section keyword determination unit 250 selects, as an audio-based keyword, one of the keywords existing in the keyword table 244 from the audio-based text. The section keyword determination unit 250 confirms whether there is a keyword that matches the audio-based text in the keyword table generation unit 242 previously generated by the keyword table generation unit 242.

When there are one or more keywords (e.g., Kocher's Point, 2% Lidocaine, and No. 20 Blade) matched with the audio-based text in the previously generated keyword table 244, the section keyword determination unit 250 selects the corresponding keyword as the audio-based keyword. For example, when there are two keywords matched with the audio-based text in the previously generated keyword table 244, the section keyword determination unit 250 selects both of the two keywords (e.g., Kocher's Point, 2% Lidocaine, and No. 20 Blade) as the audio-based keywords.

FIG. 6 is a view showing control of each section of a stream image according to the present embodiment.

When the audio-based text corresponding to the audio of the stream image matches the keywords existing in the keyword table 244, the section keyword determination unit 250 selects the keyword as an audio-based keyword (e.g., No. 20 Blade). The section control unit 260 confirms a time when the audio-based keyword (e.g., No. 20 Blade) is selected from the stream image. The section control unit 260 may set a specific section from the time when the audio-based keyword (e.g., No. 20 Blade) is selected to a frame having an object (e.g., No. 20 Blade image) matched to the keyword.

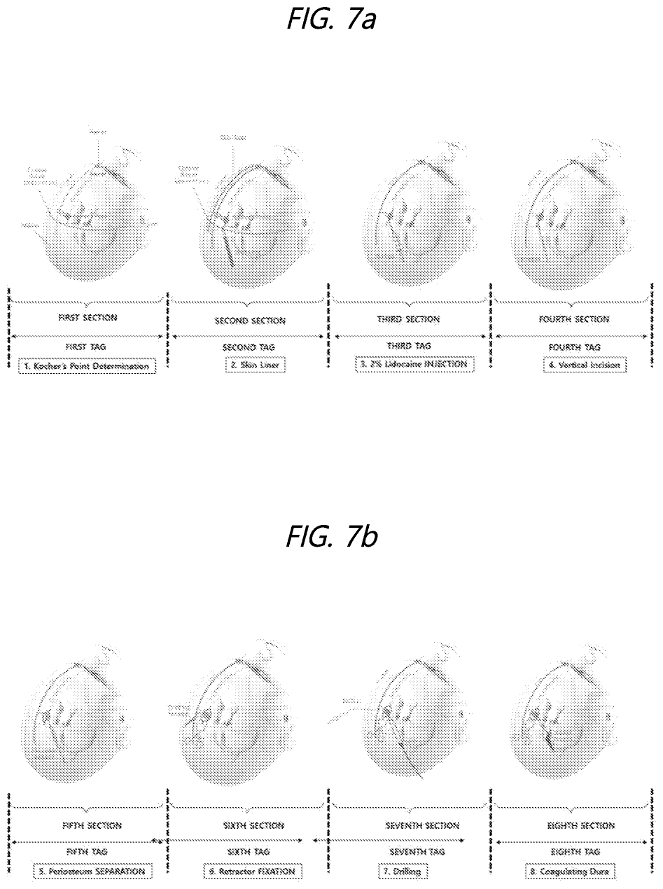

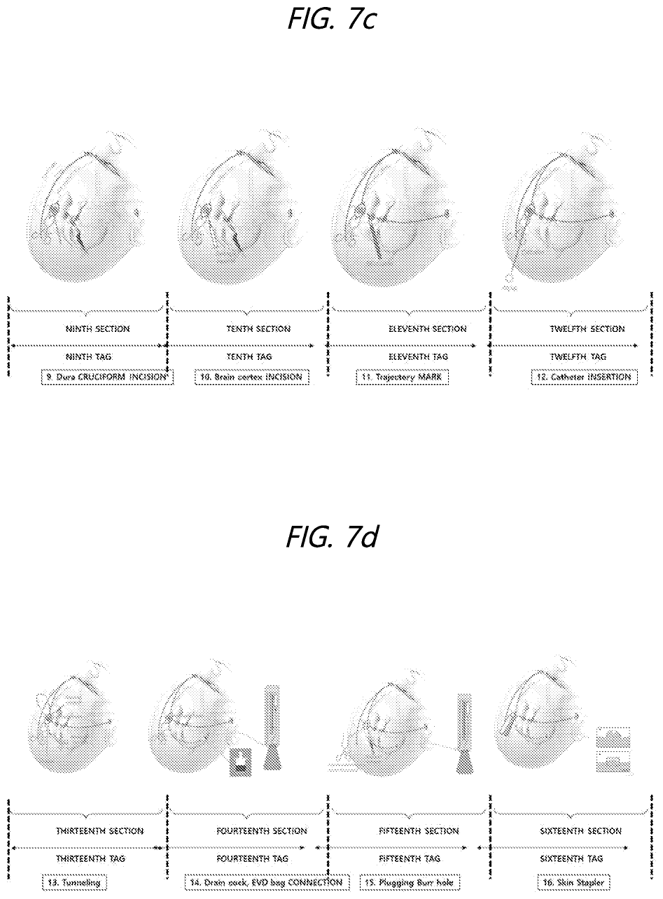

FIGS. 7a to 7d are views showing tags corresponding to scenes of a reference image according to the present embodiment.

The reference image section division unit 214 divides the reference image into a plurality of sections. For example, the reference image section division unit 214 divides an external ventricular drainage (EVD) surgery image into first to sixteenth sections when the reference image is EVD surgery.

The reference image section division unit 214 provides the first section corresponding to attachment of a marker at a 1 cm point from Nasion and EAM anterior, in order to mark a guide of a position where a catheter is inserted in the EVD surgery image (reference image). Thereafter, the reference tag insertion unit 216 for each section inserts a first tag representing Kocher's point determination into the first section.

The reference image section division unit 214 provides the second section for a process of dressing after marking a vertical incision line in the EVD surgery image (reference image). Thereafter, the reference tag insertion unit 216 for each section inserts a second tag representing a skin liner into the second section.

The reference image section division unit 214 provides the third section for injecting anesthesia into a lidocaine incision position and a subcutaneous tissue in the EVD surgical image (reference image). Thereafter, the reference tag insertion unit 216 for each section inserts a third tag representing 2% lidocaine injection into the third section.

The reference image section division unit 214 provides the fourth section for a vertical incision process using No. 20 blade in the EVD surgical image (reference image). Thereafter, the reference tag insertion unit 216 for each section inserts a fourth tag representing vertical incision into the fourth section.

The reference image section division unit 214 provides the fifth section for a process of dissecting periosteal with a periosteal elevator in the EVD surgical image (reference image). Thereafter, the reference tag insertion unit 216 for each section inserts a fifth tag representing periosteum separation into the fifth section.

The reference image section division unit 214 provides the sixth section for a process of fixing an incision site using toothed forceps and a retractor in the EVD surgery image (reference image). Thereafter, the reference tag insertion unit 216 for each section inserts a sixth tag representing fixation using the retractor into the sixth section.

The reference image section division unit 214 provides the seventh section for a drilling process in the EVD surgical image (reference image). Thereafter, the reference tag insertion unit 216 for each section inserts a seventh tag representing drilling into the seventh section.

The reference image section division unit 214 provides the eighth section for a process of coagulating a dura using bipolar forceps in the EVD surgery image (reference image). Thereafter, the reference tag insertion unit 216 for each section inserts an eighth tag representing coagulating dura into the eighth section.

The reference image section division unit 214 provides the ninth section for a process of making incision of a dura into a cruciform (+) using No. 15 Blade and coagulating the incised dura using the bipolar forceps in the EVD surgery image (reference image). Thereafter, the reference tag insertion unit 216 for each section inserts a ninth tag representing dura cruciform incision into the ninth section.

The reference image section division unit 214 provides the tenth section for a process of making small incision of a brain cortex in a cruciform (+) using a syringe needle to form an opening through which the catheter may enter and coagulating a cortex opening site using the bipolar forceps, in the EVD surgery image (reference image). Thereafter, the reference tag insertion unit 216 for each section inserts a tenth tag representing a brain cortex into the tenth section.

The reference image section division unit 214 provides the eleventh section for a process of marking a trajectory in the EVD surgical image (reference image). Thereafter, the reference tag insertion unit 216 for each section inserts an eleventh tag representing a trajectory mark into the eleventh section.

The reference image section division unit 214 provides the twelfth section for a process of inserting the catheter at a depth of 5 cm, removing a stylet, and confirming whether a cerebrospinal fluid (CSF) is discharged, in the EVD surgery image (reference image). Thereafter, the reference tag insertion unit 216 for each section inserts a twelfth tag representing catheter insertion into the twelfth section. The reference image section division unit 214 provides the thirteenth section for a process of finely incising the skin with No. 15 Blade to form an outlet to be tunneled to a subcutaneous layer by 4 cm to 5 cm, holding the catheter using bayonet forceps, and taking a catheter distal part out of the skin using mosquito forceps, in the EVD surgery image (reference image). Thereafter, the reference tag insertion unit 216 for each section inserts a thirteenth tag representing tunneling into the thirteenth section.

The reference image section division unit 214 provides the fourteenth section for a process of connecting a drain cock and an EVD bag to a catheter distal end in the EVD surgery image (reference image). Thereafter, the reference tag insertion unit 216 for each section inserts a fourteenth tag representing EVD bag connection into the fourteenth section.

The reference image section division unit 214 provides the fifteenth section for a process of plugging a burr hole with a gelfoam having a size of 1.5 cm×1.5 cm in the EVD surgery image (reference image). Thereafter, the reference tag insertion unit 216 for each section inserts a fifteenth tag representing a plugging burr hole into the fifteenth section.

The reference image section division unit 214 provides the sixteenth section for a process of plugging a burr hole with a gelfoam having a size of 1.5 cm×1.5 cm in the EVD surgery image (reference image). Thereafter, the reference tag insertion unit 216 for each section inserts a sixteenth tag representing skin stapler into the sixteenth section.

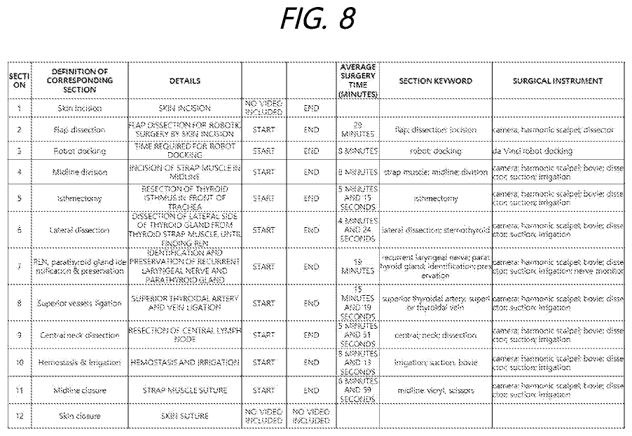

FIG. 8 is a view showing an influence table (proficiency evaluation table) according to the present embodiment.

The keyword table 244 stores a section of surgery in which a surgical resection range is set by single lob resection while dividing the section of surgery into a first section (skin incision), a second section (flap dissection), a third section (robot docking), a fourth section (midline division), a fifth section (isthmectomy), a sixth section (lateral dissection), a seventh section (RNL, parathyroid gland identification & preservation), an eighth section (superior vessels ligation), a ninth section (central neck dissection), a tenth section (hemostasis & irrigation), an eleventh section (midline closure), and a twelfth section (skin closure). The keyword table 244 may change the first to twelfth sections according to an operator or a type of surgery.

The keyword table 244 may store the sixth section (lateral dissection), the seventh section (RNL, parathyroid gland identification & preservation), and the eighth section (superior vessels ligation) in combination according to a request of a person who made settings.

The keyword table 244 may store proficiency evaluation indices for the single lobe resection as one or more of the surgery time, the surgical instrument, the section keyword, and the occurrence of surgical complications.

The keyword table 244 stores the second section (flap dissection) of the single lob resection while matching the average surgery time to 29 minutes, the section keyword to flap, dissection, and incision, and the surgical instrument to camera, harmonic scalpel, and dissector.

The keyword table 244 stores the third section (robot docking) of the single lob resection while matching the average surgery time to 8 minutes, the section keyword to robot and docking, and the surgical instrument to da Vinci robot docking.

The keyword table 244 stores the fourth section (midline division) of the single lob resection while matching the average surgery time to 8 minutes, the section keyword to strap muscle, midline, and division, and the surgical instrument to camera, harmonic scalpel, bovie, dissector, suction, and irrigation.

The keyword table 244 stores the fifth section (isthmectomy) of the single lob resection while matching the average surgery time to 5 minutes and 15 seconds, the section keyword to isthmectomy, and the surgical instrument to camera, harmonic scalpel, bovie, dissector, suction, and irrigation.

The keyword table 244 stores the sixth section (lateral dissection) of the single lob resection while matching the average surgery time to 4 minutes and 24 seconds, the section keyword to lateral dissection and sternothyroid, and the surgical instrument to camera, harmonic scalpel, bovie, dissector, suction, and irrigation.

The keyword table 244 stores the seventh section (RLN, parathyroid gland identification & preservation) of the single lob resection while matching the average surgery time to 19 minutes, the section keyword to recurrent laryngeal nerve, parathyroid gland, identification, and preservation, midline, and the surgical instrument to camera, harmonic scalpel, bovie, dissector, suction, irrigation, and nerve monitor.

The keyword table 244 stores the eighth section (superior vessels ligation) of the single lob resection while matching the average surgery time 15 minutes and 19 seconds, the section keyword to superior thyroidal artery and superior thyroidal vein, and the surgical instrument to camera, harmonic scalpel, bovie, dissector, suction, and irrigation.

The keyword table 244 stores the ninth section (central neck dissection) of the single lob resection while matching the average surgery time to 5 minutes and 51 seconds, the section keyword to central, neck, and dissection, and the surgical instrument to camera, harmonic scalpel, bovie, dissector, suction, and irrigation.

The keyword table 244 stores the tenth section (hemostasis & irrigation) of the single lob resection while matching the average surgery time to 18 minutes and 13 seconds, the section keyword to irrigation, suction, and bovie, and the surgical instrument to camera, harmonic scalpel, bovie, dissector, suction, and irrigation.

The keyword table 244 stores the eleventh section (midline closure) of the single lob resection while matching the average surgery time to 6 minutes and 59 seconds, the section keyword to midline, vicryl, and scissors, and the surgical instrument to camera, harmonic scalpel, bovie, dissector, suction, and irrigation.

The proficiency evaluation unit 280 confirms a section keyword matched to each of the plurality of sections for the stream image of the single lobe resection, which is divided into the first to twelfth sections. The proficiency evaluation unit 280 extracts proficiency evaluation indices (surgery duration of time, surgical instrument, section keyword, and occurrence of surgical complications, etc.) for each of the first to twelfth sections of the single lob resection.

The proficiency evaluation unit 280 generates proficiency evaluation data according to satisfaction of the proficiency evaluation indices for each of the first to twelfth sections of the single lob resection. The proficiency evaluation unit 280 gives a proficiency evaluation score, which is obtained by evaluating the proficiency, according to the satisfaction of the proficiency evaluation indices for the first to twelfth sections of the single lobe resection, and calculates a final proficiency evaluation score by finally summing up scores for each of the plurality of sections.

The proficiency evaluation unit 280 extracts one or more of the surgery duration of time, the surgical instrument, the section keyword, and the occurrence of surgical complications as the proficiency evaluation indices for each of the first to twelfth sections of the single lob resection.

When there is a keyword that matches flap, dissection, and incision, which are the section keywords pre-stored in the keyword table 244 for the second section among audio-based keywords or scene-based keywords extracted from the second section for the stream image of the single lobe resection, the proficiency evaluation unit 280 gives the medical person who performs surgery in the second section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the second section for the stream image of the single lob resection is within 29 minutes, which is the average surgery time pre-stored in the keyword table 244 for the second section.

The proficiency evaluation unit 280 selects a keyword coincident with the audio-based keyword or scene-based keyword extracted from the second section for the stream image of the single lob resection among flap, dissection, and incision, which are the section keywords pre-stored in the keyword table 244 for the second section. The proficiency evaluation unit 280 extracts an image object matched to the keyword coincident with the audio-based keyword or the scene-based keyword. The proficiency evaluation unit 280 gives the medical person who performs surgery in the second section for a score, which is equal to or higher than the preset threshold, as an evaluation score for the corresponding section when there is an object coincident with camera, harmonic scalpel, and dissector, which are the surgical instruments pre-stored in the keyword table 244 for the second section among image objects.

When there is a keyword that matches robot and docking, which are the section keywords pre-stored in the keyword table 244 for the third section among the audio-based keywords or scene-based keywords extracted from the third section for the stream image of the single lobe resection, the proficiency evaluation unit 280 gives the medical person who performs surgery in the third section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the third section for the stream image of the single lob resection is within 8 minutes, which is the average surgery time pre-stored in the keyword table 244 for the third section.

The proficiency evaluation unit 280 selects a keyword coincident with the audio-based keyword or scene-based keyword extracted from the third section for the stream image of the single lob resection among robot and docking, which are the section keywords pre-stored in the keyword table 244 for the third section. The proficiency evaluation unit 280 extracts an image object matched to the keyword coincident with the audio-based keyword or the scene-based keyword. The proficiency evaluation unit 280 gives the medical person who performs surgery in the third section for a score, which is equal to or higher than the preset threshold, as an evaluation score for the corresponding section when there is an object coincident with Vinci robot docking, which is the surgical instrument pre-stored in the keyword table 244 for the third section among image objects.

When there is a keyword that matches strap muscle, midline, and division, which are the section keywords pre-stored in the keyword table 244 for the fourth section among the audio-based keywords or scene-based keywords extracted from the fourth section for the stream image of the single lobe resection, the proficiency evaluation unit 280 gives the medical person who performs surgery in the fourth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the fourth section for the stream image of the single lob resection is within 8 minutes, which is the average surgery time pre-stored in the keyword table 244 for the fourth section.

The proficiency evaluation unit 280 selects a keyword coincident with the audio-based keyword or scene-based keyword extracted from the fourth section for the stream image of the single lob resection among strap muscle, midline, and division, which are the section keywords pre-stored in the keyword table 244 for the fourth section. The proficiency evaluation unit 280 extracts an image object matched to the keyword coincident with the audio-based keyword or the scene-based keyword. The proficiency evaluation unit 280 gives the medical person who performs surgery in the fourth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the fourth section of the keyword table 244 among the image objects.

When there is a keyword that matches isthmectomy, which is the section keyword pre-stored in the keyword table 244 for the fifth section among the audio-based keywords or scene-based keywords extracted from the fifth section for the stream image of the single lobe resection, the proficiency evaluation unit 280 gives the medical person who performs surgery in the fifth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the fifth section for the stream image of the single lob resection is within 5 minutes and 15 seconds, which is the average surgery time pre-stored in the keyword table 244 for the fifth section.

The proficiency evaluation unit 280 selects a keyword coincident with isthmectomy, which is the section keyword pre-stored in the keyword table 244 for the fifth section, and the audio-based keyword or scene-based keyword extracted from the fifth section for the stream image of the single lob resection. The proficiency evaluation unit 280 extracts an image object matched to the keyword coincident with the audio-based keyword or the scene-based keyword. The proficiency evaluation unit 280 gives the medical person who performs surgery in the fifth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the fifth section of the keyword table 244 among the image objects.

When there is a keyword that matches lateral dissection, and sternothyroid, which are the section keywords pre-stored in the keyword table 244 for the sixth section among the audio-based keywords or scene-based keywords extracted from the sixth section for the stream image of the single lobe resection, the proficiency evaluation unit 280 gives the medical person who performs surgery in the sixth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the sixth section for the stream image of the single lob resection is within 4 minutes and 24 seconds, which is the average surgery time pre-stored in the keyword table 244 for the sixth section.

The proficiency evaluation unit 280 selects a keyword coincident with the audio-based keyword or scene-based keyword extracted from the sixth section for the stream image of the single lob resection among lateral dissection and sternothyroid, which are the section keywords pre-stored in the keyword table 244 for the sixth section. The proficiency evaluation unit 280 extracts an image object matched to the keyword coincident with the audio-based keyword or the scene-based keyword. The proficiency evaluation unit 280 gives the medical person who performs surgery in the sixth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the sixth section of the keyword table 244 among the image objects.

When there is a keyword that matches recurrent laryngeal nerve, parathyroid gland, identification, and preservation, which are the section keywords pre-stored in the keyword table 244 for the seventh section among the audio-based keywords or scene-based keywords extracted from the seventh section for the stream image of the single lobe resection, the proficiency evaluation unit 280 gives the medical person who performs surgery in the seventh section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the seventh section for the stream image of the single lob resection is within 19 minutes, which is the average surgery time pre-stored in the keyword table 244 for the seventh section.

The proficiency evaluation unit 280 selects a keyword coincident with the audio-based keyword or scene-based keyword extracted from the seventh section for the stream image of the single lob resection among recurrent laryngeal nerve, parathyroid gland, identification, and preservation, which are the section keywords pre-stored in the keyword table 244 for the seventh section. The proficiency evaluation unit 280 extracts an image object matched to the keyword coincident with the audio-based keyword or the scene-based keyword. The proficiency evaluation unit 280 gives the medical person who performs surgery in the seventh section for a score, which is equal to or higher than the preset threshold, as an evaluation score for the corresponding section when there is an object coincident with the camera, harmonic scalpel, bovie, dissector, suction, irrigation, and nerve monitor, which are the surgical instruments pre-stored in the seventh section of the keyword table 244 among the image objects.

When there is a keyword that matches superior thyroidal artery and superior thyroidal vein, which are the section keywords pre-stored in the keyword table 244 for the eighth section among the audio-based keywords or scene-based keywords extracted from the eighth section for the stream image of the single lobe resection, the proficiency evaluation unit 280 gives the medical person who performs surgery in the eighth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the eighth section for the stream image of the single lob resection is within 15 minutes and 19 seconds, which is the average surgery time pre-stored in the keyword table 244 for the eighth section.

The proficiency evaluation unit 280 selects a keyword coincident with the audio-based keyword or scene-based keyword extracted from the eighth section for the stream image of the single lob resection among superior thyroidal artery and superior thyroidal vein, which are the section keywords pre-stored in the keyword table 244 for the eighth section. The proficiency evaluation unit 280 extracts an image object matched to the keyword coincident with the audio-based keyword or the scene-based keyword. The proficiency evaluation unit 280 gives the medical person who performs surgery in the eighth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the eighth section of the keyword table 244 among the image objects.

When there is a keyword that matches central, neck, and dissection, which are the section keywords pre-stored in the keyword table 244 for the ninth section among the audio-based keywords or scene-based keywords extracted from the ninth section for the stream image of the single lobe resection, the proficiency evaluation unit 280 gives the medical person who performs surgery in the ninth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the ninth section for the stream image of the single lob resection is within 5 minutes and 51 seconds, which is the average surgery time pre-stored in the keyword table 244 for the ninth section.

The proficiency evaluation unit 280 selects a keyword coincident with the audio-based keyword or scene-based keyword extracted from the ninth section for the stream image of the single lob resection among central, neck, and dissection, which are the section keywords pre-stored in the keyword table 244 for the ninth section. The proficiency evaluation unit 280 extracts an image object matched to the keyword coincident with the audio-based keyword or the scene-based keyword. The proficiency evaluation unit 280 gives the medical person who performs surgery in the ninth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the ninth section of the keyword table 244 among the image objects.

When there is a keyword that matches irrigation, suction, and bovie, which are the section keywords pre-stored in the keyword table 244 for the tenth section among the audio-based keywords or scene-based keywords extracted from the tenth section for the stream image of the single lobe resection, the proficiency evaluation unit 280 gives the medical person who performs surgery in the tenth section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the tenth section for the stream image of the single lob resection is within 18 minutes and 13 seconds, which is the average surgery time pre-stored in the keyword table 244 for the tenth section.

The proficiency evaluation unit 280 selects a keyword coincident with the audio-based keyword or scene-based keyword extracted from the tenth section for the stream image of the single lob resection among irrigation, suction, and bovie, which are the section keywords pre-stored in the keyword table 244 for the tenth section. The proficiency evaluation unit 280 extracts an image object matched to the keyword coincident with the audio-based keyword or the scene-based keyword. The proficiency evaluation unit 280 gives the medical person who performs surgery in the tenth section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the tenth section of the keyword table 244 among the image objects.

When there is a keyword that matches midline, vicryl, and scissors, which are the section keywords pre-stored in the keyword table 244 for the eleventh section among the audio-based keywords or scene-based keywords extracted from the tenth section for the stream image of the single lobe resection, the proficiency evaluation unit 280 gives the medical person who performs surgery in the eleventh section for a score, which is equal to or higher than a preset threshold, as an evaluation score of the corresponding section.

The proficiency evaluation unit 280 gives the medical person who performs surgery in the corresponding section for a score, which is equal to or higher than a preset threshold, as an evaluation score of the corresponding section when the surgery duration of time extracted from the eleventh section for the stream image of the single lob resection is within 6 minutes and 59 seconds, which is the average surgery time pre-stored in the keyword table 244 for the eleventh section.

The proficiency evaluation unit 280 selects a keyword coincident with the audio-based keyword or scene-based keyword extracted from the eleventh section for the stream image of the single lob resection among midline, vicryl, and scissors, which are the section keywords pre-stored in the keyword table 244 for the eleventh section. The proficiency evaluation unit 280 extracts an image object matched to the keyword coincident with the audio-based keyword or the scene-based keyword. The proficiency evaluation unit 280 gives the medical person who performs surgery in the eleventh section for a score, which is equal to or higher than the preset threshold, as an evaluation score for the corresponding section when there is an object coincident with the camera, harmonic scalpel, bovie, dissector, suction, and irrigation, which are the surgical instruments pre-stored in the eleventh section of the keyword table 244 among the image objects.

The above description is merely illustrative of the technical idea of the present embodiment, and those skilled in the art to which this embodiment belongs will appreciate that various modifications and variations are possible without departing from the essential characteristics of the embodiments. Therefore, the present embodiments are not intended to limit the technical idea of the present disclosure, and the scope of the technical idea of the present disclosure is not limited by these embodiments. Accordingly, the scope of protection sought for by the present invention should be interpreted by the claims below, and all technical ideas within the scope equivalent thereto should be interpreted as being included in the scope of rights of the present invention.

1. An apparatus for evaluating proficiency, the apparatus comprising:
a keyword table that stores proficiency evaluation indices preset for each type of surgery;
an image stream unit that receives a stream image for a specific surgery;
a stream image section division unit that divides the stream image into a plurality sections; and
a proficiency evaluation unit that extracts the proficiency evaluation indices corresponding to each of the plurality of sections for the stream image divided into the plurality of sections, and generates proficiency evaluation data according to satisfaction for the proficiency evaluation indices for each of the plurality of sections.

2. The apparatus of claim 1, wherein the proficiency evaluation unit gives a proficiency evaluation score, which is obtained by evaluating proficiency according to the satisfaction for the proficiency evaluation indices for each of the plurality of sections, and calculates a final proficiency evaluation score by finally summing up scores for each of the plurality of sections.

3. The apparatus of claim 1, wherein the proficiency evaluation unit extracts one or more of a surgery duration of time, a surgical instrument, and a section keyword as the proficiency evaluation indices when proficiency is evaluated for each of the plurality of sections.

4. The apparatus of claim 3, wherein, when there is a keyword that matches the section keyword pre-stored in the keyword table for each section among audio-based keywords or scene-based keywords extracted from the stream image for each of the plurality of sections, the proficiency evaluation unit gives medical a person who performs surgery in a corresponding section for a score, which is equal to or higher than a preset threshold, as an evaluation score of the corresponding section.

5. The apparatus of claim 3, wherein, when the surgery duration of time extracted from the stream image for each of the plurality of sections is within an average surgery time pre-stored in the keyword table for each section, the proficiency evaluation unit gives a medical person who performs surgery in a corresponding section for a score, which is equal to or higher than a preset threshold, as an evaluation score of the corresponding section.

6. The apparatus of claim 3, wherein the proficiency evaluation unit
gives a medical person who performs surgery in a specific section for a maximum proficiency evaluation score corresponding to the section keyword when the surgery duration of time is within an average surgery time,
gives the medical person who performs surgery in the specific section for a proficiency evaluation score, which is obtained by subtracting a first score corresponding to the section keyword, when the surgery duration of time exceeds the average surgery time by a first threshold (e.g.: 10 seconds) or less, and
gives the medical person who performs surgery in the specific section for a proficiency evaluation score, which is obtained by subtracting a second score corresponding to the section keyword, when the surgery duration of time exceeds the average surgery time by a second threshold (e.g.: 20 seconds) or less.

7. The apparatus of claim 3, wherein the proficiency evaluation unit selects a keyword that matches an audio-based keyword or a scene-based keyword extracted from the stream image for each of the plurality of sections among section keywords pre-stored in the keyword table for each section, extracts an image object matched to the keyword coincident with the audio-based keyword or the scene-based keyword, and gives a medical person who performs surgery in a corresponding section for a score, which is equal to or higher than a preset threshold, as an evaluation score of the corresponding section when there is an object that matches the surgical instrument pre-stored in the keyword table for each section among image objects.

8. The apparatus of claim 3, wherein when a plurality of indices are satisfied among the surgery duration of time, the surgical instrument, and the section keyword, the proficient evaluation unit calculates a final proficiency evaluation score by summing up scores for each section after reflecting a weight to each satisfied index to provide an evaluation score for each section.
